(19) United States
(12) Patent Application Publication
(10) Pub. No.: US 2022/0096658 A1
(43) Pub. Date: Mar. 31, 2022

(54) ADENO-ASSOCIATED VIRUSES AND THEIR USES FOR INNER EAR THERAPY

(71) Applicants: Wade W. Chien, Bethesda, MD (US); Jean Bennett, Philadelphia, PA (US)

(72) Inventors: Wade W. Chien, Bethesda, MD (US); Jean Bennett, Philadelphia, PA (US)

(73) Assignee: The United States of America, As Represented by The Secretary, Department of Health and Human Svcs., Rockville, MD (US)

(21) Appl. No.: 17/416,311

(22) PCT Filed: Dec. 20, 2019
(86) PCT No.: PCT/US2019/068070

§ 371 Date: Jun. 18, 2021

Related U.S. Application Data
(60) Provisional application No. 62/784,306, filed on Dec. 21, 2018.

(51) Int. Cl. A61K 48/00; C12N 15/86; A61P 27/16

(52) U.S. Cl. CPC A61K 48/0075; C12N 15/86; C12N 2750/14143; C12N 2750/14122; A61P 27/16

(57) ABSTRACT

Provided herein are adeno-associated viruses and methods for using same to treat or prevent disorders that affect the inner ear of a subject.

### PRIOR RELATED APPLICATIONS

This application claims the benefit of U.S. Provisional Application No. 62/784,306 filed on Dec. 21, 2018, which is hereby incorporated by reference in its entirety.

### BACKGROUND

Hearing loss is one of the most common disabilities affecting the world's population today. According to the National Health and Nutritional Examination Survey, nearly two thirds of U.S. adults aged 70 years and older are affected by hearing loss. Hearing loss is associated with inner ear hair cell damage. Inner ear gene therapy is a promising therapeutic modality which can potentially prevent and reverse hair cell damage. Although adeno-associated viral vector (AAV)-mediated inner ear gene therapy has been applied to animal models of hereditary hearing loss to improve auditory function, infection rates in some inner ear hair cell types are low. In addition, the infection efficiency of conventional AAVs for supporting cells in the inner ear is also low. In order for inner ear gene therapy to effectively treat hearing loss, a viral vector with higher infection efficiency is required.

### BRIEF SUMMARY OF THE INVENTION

The present disclosure is directed to compositions and methods for treating or preventing diseases or disorders that affect the inner ear of a subject. The inventors have discovered that a recombinant AAV comprising a modified AAV capsid protein can infect inner ear hair cells to effectively deliver genetic material into the inner ear hair cells of a subject. In some embodiments, the compositions and methods provided herein can be used to treat or prevent hearing loss and/or dizziness in a subject.

In some embodiments, the present disclosure provides a recombinant adeno-associated virus (AAV) virion comprising: (a) a modified AAV capsid protein, wherein the modified AAV capsid protein comprises a peptide insertion relative to a corresponding parental AAV capsid protein, wherein the peptide insertion comprises the amino acid sequence LGETTRP (SEQ ID NO: 1), wherein the insertion in the modified AAV capsid protein is between amino acids corresponding to amino acids 587 and 588 of VP1 of AAV2; and (b) a heterologous nucleic acid that produces an expression product, wherein the expression product reduces hearing loss and/or dizziness.

In some embodiments, the expression product is a nucleic acid that decreases expression of a gene associated with hearing loss and/or dizziness, wherein the gene associated with hearing loss and/or dizziness is selected from the group consisting of DIAPH1, KCNQ4, GJB3, IFNLR1, GJB2, GJB6, MYH1, CEACAM16, GSDME/DFNA5. WFS1, LMX1A, TECTA, COCH, EYA4, MYO7A, COL11A2, POU4F3, MYH9, ACTG1, MYO6, SIX1, SLC17A8, REST, GRHL2, NLRP3, TMC1, COL11A1, CRYM, P2RX2, CCDC50, MIRN96, TJP2, TNC, SMAC/DIABLO, TBC1D24, CD164, OSBPL2, HOMER2, KITLG, MCM2, PTPRQ, DMXL2, MYO3A and PDE1C.

In some embodiments, the expression product is a polypeptide that reduces hearing loss and/or dizziness, wherein the polypeptide is selected from the group consisting of GJB2, GJB6, MYO7A, MYO15A, SLC26A4, TMIE, TMC1, TMPRSS3, OTOF, CDH23, GIPC3, STRC, USH1C, OTOG, TECTA, OTOA, PCDH15, RDX, GRXCR1, TRIOBP, CLDN14, MYO3A, WHRN, CDC14A, ESRRB, ESPN, MYO6, HGF, ILDR1, ADCY1, CIB2, MARVELD2, BDP1, COL11A2, PDZD7, PJVK, SLC22A4, SLC26A5, LRTOMT/COMT2, DCDC2, LHFPLS, S1PR2, PNPT1, BSND, MSRB3, SYNE4, LOXHD1, TPRN, GPSM2, PTPRQ, OTOGL, TBC1D24, ELMOD3, KARS, SERPINB6, CABP2, NARS2, MET, TSPEAR, TMEM132E, PPIP5K2, GRXCR2, EPS8, CLIC5, FAM65B, DFNB32, EPS8L2, ROR1, WBP2, ESRP1, MPZL2, PRPS1, POU3F4, SMPX, AIFM1 and COL4A.

In some embodiments, the recombinant AAV virion is selected from the group consisting of AAV2, AAV5, AAV8 and AAV9. In some embodiments, The recombinant AAV virion is an AA2 virion comprising a modified AAV2-VP1 capsid protein, for example, a AAV2.7m8 virion.

In some embodiments, the expression product is a nucleic acid that decreases expression of a gene associated with hearing loss is an interfering RNA. In some embodiments, the interfering RNA is an antisense molecule, a short interfering RNA or an miRNA.

In some embodiments, the AAV virions produce an expression product that reduces age-related hearing loss, hereditary hearing loss, noise-induced hearing loss, disease-associated hearing loss or hearing loss resulting from trauma.

In another embodiment, the present disclosure provides a method for treating or preventing inner ear hair cell damage in a subject comprising administering to the subject having inner ear hair cell damage or at risk of developing inner ear hair cell damage, an effective amount of any recombinant AAV virion described herein.

In some embodiments, the subject has or is at risk of developing age-related hearing loss, hereditary hearing loss, noise-induced hearing loss, disease-associated hearing loss and hearing loss resulting from trauma.

In some embodiments, the recombinant AAV virion infects inner hair cells and outer hair cells of the cochlea. In some embodiments, the recombinant AAV virion infects glia-like supporting cells in the cochlea. In some embodiments, the supporting cells are inner pillar cells or inner phalangeal cells.

In some embodiments, the recombinant AAV virion increases inner ear hair cell regeneration, for example, cochlear hair cell regeneration.

In another embodiment, the present disclosure also provides a method for treating or preventing hearing loss and/or dizziness in a subject, comprising administering to the subject having hearing loss and/or dizziness or at risk of developing hearing loss and/or dizziness, an effective amount of any recombinant AAV virion described herein.

In some embodiments, the subject having hearing loss or at risk of developing hearing loss is a subject that has or is at risk of developing age-related hearing loss, hereditary hearing loss, noise-induced hearing loss, disease-associated hearing loss and hearing loss resulting from trauma.

In some embodiments, the recombinant AAV virion infects inner ear hair cells of the subject that has or is at risk of developing hearing loss. In some embodiments, inner ear hair cells are inner and/or outer hair cells of the cochlea. In some embodiments, the recombinant AAV virion infects glia-like supporting cells in the cochlea of the subject. In some embodiments, the supporting cells are inner pillar cells and/or inner phalangeal cells. In some embodiments, the recombinant AAV virion increases inner ear hair cell regeneration, for example, cochlear hair cell regeneration.

In any of the methods provided herein, the recombinant AAV virion can be administered to the subject intravenously, intrathecally, intratypmanically, via round window administration, via semicircular canal delivery, or via stapedotomy. In some embodiments, the recombinant AAV virion is administered via canalostomy into the posterior semicircular canal of the subject.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIGS. 1a-1h show that AAV2.7m8 infects cochlear inner and outer hair cells with high efficiency. (a-f) When AAV2.7m8-GFP (a) was injected into neonatal mouse inner ear via the posterior semicircular canal approach, the IHCs and OHCs were infected with high efficiency throughout the entire cochlea. AAV8BP2-GFP (b) injection caused some loss in IHCs (white arrows). AAV-DJ-GFP (c) infected cochlear hair cells at very low levels. AAV2-GFP (d), AAV8-GFP (e), and Anc80L65-GFP (f) infected IHCs at high levels, but the OHC infection efficiency was less than AAV2.7m8-GFP. GFP expression is shown in green, and Myo7a expression (a marker for hair cells) is shown in red. 40× images of the cochlear apex are shown. Scale bar represents 20 μm. (g & h). Quantification of IHC (g) and OHC (h) infection efficiency. Error bars represent standard errors. Statistical significance in reference to AAV2.7m8 is shown above error bars (*represents p<0.05, ** represents p<0.01, and *** represents p<0.001). IHC: inner hair cell. OHC: outer hair cell.

FIGS. 2a-2b show that AAV2.7m8 infects inner and outer hair cells throughout the entire cochlea. 10× (a) and 40× (b) images of a mouse cochlea that underwent AAV2.7m8-GFP injection via the posterior semicircular canal approach. GFP expression is seen in both IHCs and OHCs throughout the entire cochlea. GFP expression is shown in green, and Myo7a expression (a marker for hair cells) is shown in red.

FIGS. 3a-3g show that AAV2.7m8 infects vestibular hair cells with lower efficiency. (a-f) 10× and 40× images of utricles showing hair cell infection efficiency in response to posterior canal AAV delivery. AAV2.7m8-GFP (a), AAV8BP2-GFP (b), AAV-DJ-GFP (c), AAV2-GFP (d) infected utricular hair cells at lower levels. In contrast, AAV8-GFP (e) and Anc80L65-GFP (f) infected utricular hair cells at higher levels. GFP expression is shown in green, and Myo7a expression (a marker for hair cells) is shown in red. (g) Quantification of utricular hair cell infection efficiency. Statistical significance in reference to AAV2.7m8 is shown above error bars (* represents p<0.05, ** represents p<0.01, and *** represents p<0.001). Error bars represent standard errors. SSC: superior semicircular canal. HSC: horizontal semicircular canal.

FIGS. 4a-4g show that AAV2.7m8 infects inner pillar cells and inner phalangeal cells with high efficiency. (a-f): Confocal images of cochlear apex showing inner pillar cell (IPC) and inner phalangeal cell (IPhC) infection efficiency in response to posterior canal AAV delivery. AAV2.7m8-GFP (a) infects the IPCs and IPhCs at high levels. In contrast, AAV8BP2 (b) does not infect IPCs and IPhCs. AAV-DJ-GFP (c), AAV2-GFP (d), AAV8-GFP (e) and Anc80L65-GFP (f) infect the IPCs at lower levels but do not infect IPhCs. (g) Quantification of IPC infection efficiency. Statistical significance in reference to AAV2.7m8 is shown above error bars (* represents p<0.05, ** represents p<0.01, and *** represents p<0.001). Error bars represent standard errors.

FIGS. 5a-5g show that AAV2.7m8 infects inner pillar cells and inner phalangeal cells with high efficiency. (a-e) Representative whole mount images of the cochlear apex from a mouse that underwent AAV2.7m8-GFP injection via the posterior semicircular canal approach. The inner pillar cells and inner phalangeal cells showed high levels of GFP expression. GFP expression is shown in green, Myo7a expression (a marker for hair cells) is shown in red, acetylated tubulin expression is shown in magenta (a marker for supporting cells), and Hoechst stain is shown in blue (marker for nuclei). 40× images are shown. (f) Orthogonal projection of the same image showing inner pillar cells and inner phalangeal cells with robust GFP expression. Images of the cochlear apex are shown. (g & h) Quantification of inner pillar cell (g) and inner phalangeal cell (h) infection efficiency. Error bars represent standard errors. IHC: inner hair cell. OHC: outer hair cell. IPC: inner pillar cell. IPhC: inner phalangeal cell.

FIGS. 6a-6b show that AAV2.7m8 has minimal adverse effect on auditory and vestibular functions in injected mice. (a) Auditory brainstem responses (ABR) were recorded to assess the auditory function of mice that underwent synthetic AAV injection via the posterior semicircular canal approach. AAV2.7m8, AAV-DJ, AAV2, AAV8, and Anc80L65 had minimal adverse effect on the auditory function, while injection of AAV8BP2 caused a 10-25 dB ABR threshold hold elevation compared to non-injected control mice. (b) Circling behavior was assessed in mice that underwent AAV injection via the posterior semicircular canal approach. AAV2.7m8, AAV-DJ, AAV2, AAV8, and Anc80L65 did not cause statistically significant increase in circling behavior compared to non-injected control mice, while injection of AAV8BP2 caused a slight elevation in circling behavior compared to non-injected control mice. Statistical significance in reference to non-injected normal control mice is shown above error bars (* represents p<0.05, ** represents p<0.01, and *** represents p<0.001). Error bars represent standard errors.

FIGS. 7a-7b show that AAV8BP2 causes inflammation in the cochlea. (a) Examination of the cochlea after AAV2.7m8-GFP injection using hematoxylin and eosin (H&E) stain showed no evidence of inflammatory cell infiltration. (b) In contrast, infiltration of inflammatory cells was seen in the cochlea after AAV8BP2 injection. SV: scala vestibuli. SM: scala media. ST: scala tympani.

FIGS. 8a-8c show that AAV8BP2 does not cause hearing loss and increased circling at lower concentration. (a) Posterior canal injection of AAV8BP2 at 0.5×10¹⁰ G.C. (AAV8BP2 0.5e¹⁰) caused no ABR threshold elevation compared to non-injected control mice. (b) Posterior canal injection of AAV8BP2 at 0.5×10¹⁰ G.C. (AAV8BP2 0.5e¹⁰) caused no elevation in circling behavior compared to non-injected control mice. (c) The infection efficiency of IHCs and OHCs is lower when AAV8BP2 is delivered at 0.5×10¹⁰ G.C. compared to 1×10¹⁰ G.C. Images taken from cochlear apex.

### DETAILED DESCRIPTION OF THE INVENTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

The inventors have discovered that a recombinant AAV comprising a modified AAV capsid protein can be used to infect the inner ear hair cells of a subject and effectively deliver genetic material into the inner ear hair cells of a subject. Provided herein are compositions and methods for treating or preventing inner ear hair cell damage. In some embodiments, the compositions and methods provided herein can be used to treat or prevent diseases or disorders that affect the inner ear of the subject.

In some embodiments, the recombinant AAV virion comprises (a) a modified AAV capsid protein, wherein the modified AAV capsid protein comprises a peptide insertion relative to a corresponding parental AAV capsid protein, wherein the peptide insertion comprises the amino acid sequence LGETTRP (SEQ ID NO: 1), wherein the insertion in the modified AAV capsid protein is between amino acids corresponding to amino acids 587 and 588 of VP1 of AAV2.

In some embodiments, the recombinant AAV virion comprises (a) a modified AAV capsid protein, wherein the modified AAV capsid protein comprises a peptide insertion relative to a corresponding parental AAV capsid protein, wherein the peptide insertion comprises the amino acid sequence LGETTRP (SEQ ID NO: 1), wherein the insertion in the modified AAV capsid protein is between amino acids corresponding to amino acids 587 and 588 of VP1 of AAV2; and (b) a heterologous nucleic acid sequence that produces an expression product, wherein the expression product reduces hearing loss and/or dizziness.

The insertion can be between amino acids 587 and 588 of AAV2, or the corresponding positions of the capsid subunit of another AAV serotype. One of skill in the art could readily align the amino acid sequence of AAV2-VP1 with the amino acid sequence of a VP1 amino acid sequence of another AAV serotype to identify amino acids corresponding to amino acids 577 and 588 of AAV2-VP1 in a VP1 from another AAV serotype, for example, in a VP1 from AAV1, AAV3, AAV4, AAV5, AAV6, AAV7, AAV8, AAV9 or AAV10. The insertion can also be made between two adjacent amino acids in amino acids corresponding to amino acids 570-611 of AAV2-VP1 or the corresponding positions of the capsid subunit of another AAV serotype.

The peptide insertion comprising LGETTRP (SEQ ID NO: 1) can be between 7 and 15 amino acids in length. For example, the insertion can be between 7 and 10 amino acids in length, between 7 and 111 amino acids in length, between 7 and 12 amino acids in length, between 7 and 13 amino acids in length, between 7 and 14 amino acids in length or between 7 and 15 amino acids in length. The insertion can also be about 7, 8, 9, 10, 11, 12, 13, 14 or 15 amino acids in length. The amino acid sequence for AAV2 VP1 capsid protein can be found under GenBank Accession No. YP_680426.1 (SEQ ID NO: 2). In some embodiments, the insertion can be made between two adjacent amino acids corresponding to amino acids 570-611, for example, between amino acids 587 and 588, of an amino acid sequence having at least 75%, 80%, 90%, 95% or 99% identity to the amino acid sequence of AAV2-VP1.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

In some embodiments, the recombinant AAV virion is an AAV2.7m8 virion. See, for example, Dalkara, D., et al. (In vivo-directed evolution of a new adeno-associated virus for therapeutic outer retinal gene delivery from the vitreous. Sci Transl Med 5, 189ra176 (2013)) and U.S. Pat. No. 9,193,956, hereby incorporated in their entireties by this reference. In some embodiments, the recombinant AAV virion provides for increased infectivity of an inner ear hair cell, for example a cochlear hair cell, as compared to the infectivity of the inner ear hair cell by a recombinant AAV virion comprising the corresponding parental AAV capsid protein that does not have a peptide insertion in the AAV VP1 capsid protein. In some embodiments, the recombinant AAV virion, for example, AAV2.7m8, provides for increased infectivity of an inner ear hair cell, for example a cochlear hair cell, as compared to the infectivity of the inner ear hair cell by a recombinant AAV8BP2 virion or a recombinant AAV Anc80L65 virion. Increased infectivity of an inner ear hair cell after administration of a recombinant AAV virion described herein, for example, AAV2.7m8, can be at least about a 5%, 10%, 20%, 30%, 40%, 50%, 60%, 70%, 80%, 90%, 100% increase or greater as compared to a control. The increase can also be at least a 2-fold, 5-fold, 10-fold, 20-fold, 30-fold, 40-fold, 50-fold increase or greater.

As used herein, a recombinant AAV virion is a viral particle comprising at least one AAV capsid protein and an encapsidated recombinant AAV vector. As used herein, a “recombinant AAV vector” refers to an AAV vector comprising a nucleic acid sequence that is not normally present in AAV (i.e., a polynucleotide heterologous to AAV), for example, a nucleic acid sequence of interest for genetic transformation of a cell. In general, the heterologous nucleic acid is flanked by at least one, and generally by two, AAV inverted terminal repeat sequences (ITRs). The term recombinant AAV vector encompasses both rAAV vector particles and recombinant AAV vector plasmids. A recombinant AAV vector may either be single-stranded (ssAAV) or self-complementary (scAAV).

The genomic sequences of various serotypes of AAV, as well as the sequences of the native terminal repeats (TRs), Rep proteins, and capsid subunits are known in the art. Such sequences may be found in the literature or in public databases such as GenBank. See, e.g., GenBank Accession Numbers NC_002077 (AAV-1), AF063497 (AAV-1), NC_001401 (AAV-2), AF043303 (AAV-2), NC_001729 (AAV-3), NC_001829 (AAV-4), U89790 (AAV-4), NC_006152 (AAV-5), AF513851 (AAV-7), AF513852 (AAV-8), and NC_006261 (AAV-8); the disclosures of which are incorporated by reference herein for teaching AAV nucleic acid and amino acid sequences.

An “AAV virus,” AAV virion,” “AAV viral particle,” or “recombinant AAV vector particle” refers to a viral particle composed of at least one AAV capsid protein and an encapsidated polynucleotide recombinant AAV vector. If the particle comprises a heterologous nucleic acid sequence (i.e. a nucleic acid sequence other than a wild-type AAV genome such as a transgene to be delivered to a mammalian cell), it can be referred to as a recombinant AAV vector. Thus, production of recombinant AAV particles or virion necessarily includes production of a recombinant AAV vector, as such a vector is contained within a recombinant AAV particle. Methods for producing AAV vectors and virions are known in the art. See, for example, Shin et al. “Recombinant Adeno-Associated Viral Vector Production and Purification,” Methods Mol. Biol. 798: 267-284 (2012)). Any of the AAV virions described herein can be used to infect one or more types of inner ear hair cells, including, but not limited to cochlear cells, vestibular cells, inner hairs cell of the cochlea, outer hair cells of the cochlea, glia-like supporting cells of the cochlea (for example, Hensen's cells, Deiters' cells, inner and outer pillar cells, Claudius cells and inner phalangeal cells).

As used throughout, a “corresponding parental AAV capsid protein” refers to an AAV capsid protein of the same AAV serotype, without the peptide insertion. As used herein, when describing recombinant AAV vectors or virions, the phrase “heterologous” refers to a nucleic acid sequence not naturally found in wild-type AAV. For example, a heterologous nucleic acid sequence that produces an expression product is a nucleic acid not normally found in a wild-type AAV. In embodiments where the heterologous nucleic acid sequence encodes a polypeptide, the encoded polypeptide is a heterologous polypeptide not normally encoded or expressed by a naturally-occurring, wild-type AAV.

As used throughout, an “expression product” is a nucleic acid sequence or a polypeptide that is expressed or produced in a cell, for example, an inner ear hair cell, after infection by an AAV virion. The expression product can be expressed by infecting cells in vitro, in vivo or ex vivo. As used in this specification and the appended claims, the singular forms “a,” “an,” and “the” include plural reference unless the context clearly dictates otherwise. Therefore, the terms “a virion” or “a cell” also refer to more than one virion or cell, for example, populations of virions or cells.

Expression products include, but are not limited to, a polypeptide, an aptamer, an antisense molecule, an interfering RNA or an mRNA. In some embodiments, the expression product is an interfering RNA selected from the group consisting of an short interfering RNA (siRNA), a short hairpin (shRNA) and an miRNA.

As used throughout, the term “nucleic acid” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

In some embodiments, a nucleic acid sequence encoding an expression product of interest is operably linked to a constitutive promoter. In other embodiments, a nucleic acid sequence encoding an expression product of interest is operably linked to an inducible promoter. In some instances, a nucleic acid sequence encoding an expression product of interest is operably linked to a tissue-specific or cell type-specific regulatory element. For example, in some instances, a nucleic acid sequence encoding an expression product of interest is operably linked to an inner ear hair cell-specific regulatory element e.g., a regulatory element that confers selective expression of the operably linked nucleic acid in an inner ear hair cell. See, for example, Boeda and Petit “A specific promoter of the sensory cells of the inner ear defined by transgenesis” Hum Mol. Genet. 19(15): 1581-9 (2001), for expression of a gene product under the control of the MYO7A promoter in inner ear hair cells. As used herein, specific expression does not mean that the expression product is expressed only in a specific tissue(s) or cell type(s), but refers to expression substantially limited to specific tissue(s) or cell types(s). Any heterologous nucleic acid that produces an expression product can further comprise a nucleic acid encoding a detectable polypeptide, for example, a fluorescent polypeptide (GFP, RFP etc.) or an active fragment thereof.

Upon infection of an inner ear hair cell of a subject with any of the AAV virions described herein, the expression product produced in the inner ear hair cell reduces hearing loss and/or dizziness in the subject. In some embodiments, the expression product is a nucleic acid sequence, for example, an antisense molecule or an interfering RNA, that decreases expression of a gene associated with hearing loss and/or dizziness in a subject.

In some embodiments, a nucleic acid sequence, for example, an antisense molecule or an interfering RNA, decreases expression of one or more genes selected from the group consisting of DIAPH1, KCNQ4, GJB3, IFNLR1, GJB2, GJB6, MYH1, CEACAM16, GSDME/DFNA5, WFS1, LMX1A, TECTA, COCH, EYA4, MYO7A, COL11A2, POU4F3, MYH9, ACTG1, MYO6, SIX1, SLC17A8, REST, GRHL2, NLRP3, TMC1, COL11A1, CRYM, P2RX2, CCDC50, MIRN96, TJP2, TNC, SMAC/DIABLO, TBC1D24, CD164, OSBPL2, HOMER2, KITLG, MCM2, PTPRQ, DMXL2, MYO3A and PDE1C in an inner ear hair cell of subject. In some embodiments, a decrease in expression is a decrease in transcription of mRNA and/or a decrease in translation of a polypeptide or a fragment thereof translated from an mRNA. The decrease or reduction in expression can be a decrease or reduction of about 5%, 10%, 20%, 30%, 40%, 50%, 60%, 70%, 80%, 90%, 100% or any percentage in between these percentages as compared to a control. By reducing expression of one or more genes selected from the group consisting of DIAPH1, KCNQ4, GJB3, IFNLR1, GJB2, GJB6, MYH1, CEACAM16, GSDME/DFNA5. WFS1, LMX1A, TECTA, COCH, EYA4, MYO7A, COL11A2, POU4F3, MYH9, ACTG1, MYO6, SIX1, SLC17A8, REST, GRHL2, NLRP3, TMC1, COL11A1, CRYM, P2RX2, CCDC50, MIRN96, TJP2, TNC, SMAC/DIABLO, TBC1D24, CD164, OSBPL2, HOMER2, KITLG, MCM2, PTPRQ, DMXL2, MYO3A and PDE1C, hearing loss can be reduced or improved.

In some embodiments, the expression product is a polypeptide that reduces or improves hearing loss and/or dizziness in a subject. As used throughout, “polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds. Fragments of any of the polypeptides described herein are also encompassed by these terms.

In some embodiments, one or more polypeptides selected from the group consisting of GJB2, GJB6, MYO7A, MYO15A, SLC26A4, TMIE, TMC1, TMPRSS3, OTOF, CDH23, GIPC3, STRC, USH1C, OTOG, TECTA, OTOA, PCDH15, RDX, GRXCR1, TRIOBP, CLDN14, MYO3A, WHRN, CDC14A, ESRRB, ESPN, MYO6, HGF, ILDR1, ADCY1, CIB2, MARVELD2, BDP1, COL11A2, PDZD7, PJVK, SLC22A4, SLC26A5, LRTOMT/COMT2, DCDC2, LHFPLS, S1PR2, PNPT1, BSND, MSRB3, SYNE4, LOXHD1, TPRN, GPSM2, PTPRQ, OTOGL, TBC1D24, ELMOD3, KARS, SERPINB6, CABP2, NARS2, MET, TSPEAR, TMEM132E, PPIP5K2, GRXCR2, EPS8, CLIC5, FAM65B, DFNB32, EPS8L2, ROR1, WBP2, ESRP1, MPZL2, PRPS1, POU3F4, SMPX, AIFM1 and COL4A or a fragment thereof are expressed in an inner ear hair cell of a subject.

In some embodiments, upon infection of an inner ear hair cell of a subject with a recombinant AAV virion described herein, there is at least a 2-fold, at least 5-fold, at least 10-fold, at least 20-fold, at least 30-fold, at least 40-fold, at least 50-fold or more than at least a 50-fold increase in the level of one or more polypeptides in the inner ear hair cell of the subject as compared to control, such that hearing loss and/or dizziness in a subject is reduced.

The expression product can be heterologous to the cell in the subject. As used herein the phrase “heterologous,” as it relates to the expression product in a cell, for example, an inner ear hair cell of the subject, refers to a nucleic acid sequence or a polypeptide not naturally found in a cell of the subject. The term “heterologous sequence” refers to a sequence not normally found in a given cell in nature. As such, a heterologous nucleotide or protein sequence may be: (a) foreign to its host cell (i.e., is exogenous to the cell); (b) naturally found in the host cell (i.e., endogenous) but present at an unnatural quantity in the cell (i.e., greater or lesser quantity than naturally found in the host cell); or (c) be naturally found in the host cell but positioned outside of its natural locus.

### Methods

Provided herein are methods for delivering a nucleic acid of interest to the inner ear by administering any of the AAV virions described herein. In some embodiments, the AAV virion comprises a nucleic acid of interest. In some embodiments, the nucleic acid of interest is delivered to inner ear hair cells, for example, cochlear cells. In some embodiments, the AAV virion is an AAV2.7m8 virion comprising the nucleic acid of interest. In some embodiments, the nucleic acid of interest decreases inner hair cell damage, reduces hearing loss and/or reduces dizziness. In some embodiments, the nucleic acid of interest encodes a polypeptide that decreases inner hair cell damage, reduces hearing loss and/or reduces dizziness.

Hearing loss is often caused by damage to inner ear hair cells, for example, cochlear hair cells. The mammalian cochlea contains two types of hair cells, inner hair cells (IHCs) and outer hair cells (OHCs), both of which are important for the detection and processing of auditory information. These hair cells are surrounded by supporting cells, a heterogeneous group of cells which are important for cochlear homeostasis. The mature mammalian hair cells are incapable of regeneration. Therefore, once the damage occurs in these cells, the degeneration process is often irreversible.

Provided herein is a method of treating or preventing inner ear hair cell damage in a subject comprising administering to the subject having inner ear hair cell damage or at risk of developing inner ear hair cell damage, an effective amount of a recombinant AAV virion described herein. In some embodiments, the recombinant virion is a recombinant AAV virion, for example, an AAV2.7m8 virion, comprising a nucleic acid sequence that decreases expression of a gene associated with inner ear hair cell damage. In some embodiments, the recombinant AAV virion is a recombinant AAV2 virion, for example, an AAV2.7m8 virion, comprising a nucleic acid sequence encoding a polypeptide that treats or prevents inner ear hair cell damage in a subject. In some embodiments, the subject having inner ear hair cell damage or at risk of developing inner ear hair cell damage, has hearing loss or is at risk of developing hearing loss. In some embodiments, the subject having inner ear hair cell damage or at risk of developing inner ear hair cell damage experiences dizziness.

In another embodiment, provided herein is a method of treating or preventing hearing loss and/or dizziness in a subject, comprising administering to the subject having hearing loss or dizziness or at risk of developing hearing loss or dizziness, an effective amount of a recombinant AAV virion described herein. In some embodiments, the recombinant AAV virion is a recombinant AAV2 virion, for example, an AAV2.7m8 virion, comprising a nucleic acid sequence that decreases expression of a gene associated with inner ear hair cell damage. In some embodiments, the recombinant virion is a recombinant AAV virion, for example, an AAV2.7m8 virion, comprising a nucleic acid sequence encoding a polypeptide that treats or prevents inner ear hair cell damage in a subject.

In some embodiments, the recombinant AAV virion increases inner ear hair cell regeneration, for example, cochlear hair cell regeneration. In some embodiments, the recombinant AAV virion infects inner hair cells and/or outer hair cells of the cochlea. In some embodiments, the recombinant AAV virion infects glia-like supporting cells in the cochlea. In some embodiments, the supporting cells infected by the recombinant AAV virion are inner pillar cells and/or inner phalangeal cells. In some embodiments, the recombinant AAV virion increases regeneration of inner hair cells, outer hair cells and/or glia-like supporting cells of the cochlea. In some embodiments, the recombinant AAV virion preferentially infects cochlear hair cells. In some embodiments, the recombinant AAV virion infection efficiency in cochlear hair cells in the inner ear of the subject is at least 5%, 10%, 20%, 30%, 40%, 50%, 60%, 70%, 80%, 90% or at least 100% higher than the recombinant AAV virion infection efficiency in vestibular cells in the inner ear of the subject. In some embodiments, the level of the expression product produced by the recombinant AAV virion in the inner ear of the subject is at least 5%, 10%, 20%, 30%, 40%, 50%, 60%, 70%, 80%, 90% or at least 100% higher in cochlear cells as compared to vestibular cells in the inner ear of the subject.

The methods and compositions provided herein can be used to treat a subject having or at risk of developing any type of hearing loss. Hearing loss can be on the level of conductivity, sensorineural and/or central level. Conductive hearing loss is caused by lesions involving the external or middle ear, resulting in the destruction of the normal pathway of airborne sound amplified by the tympanic membrane and the ossicles to the inner ear fluids. Sensorineural hearing loss is caused by lesions of the cochlea or the auditory division of the eight cranial nerve. Central hearing loss is due to lesions of the central auditory pathways. In some cases, conductive hearing loss occurs in combination with sensorineural hearing loss (mixed hearing loss).

The compositions and methods provided herein can be used to treat subjects having or at risk of developing age-related hearing loss (presbycusis), hereditary hearing loss, noise-induced hearing loss, disease-associated hearing loss, exposure to toxic substances and hearing loss resulting from trauma, to name a few.

In some embodiments, hereditary hearing loss can be caused by a mutation in one or more genes involved in hearing. Some mutations cause hearing loss that is non-syndromic, meaning that the subject does not have any other symptoms except hearing loss. Other mutations causing hearing loss are syndromic, meaning that the person has other symptoms besides hearing loss (for example, Waardenburg's syndrome, Alport's syndrome and Usher's syndrome). In some embodiments, the hereditary hearing loss is autosomal dominant hearing loss, for example, hearing loss caused by a mutation in the GJB2.

In some embodiments, a nucleic acid sequence encoding a non-mutated polypeptide of a missing or mutated gene associated with hearing loss is delivered to the inner ear hair cells of the subject to provide the inner ear hair cells with a working copy of a missing or mutated gene involved in hearing loss. In other embodiments, a nucleic acid sequence that decreases expression of a one or more mutant alleles of a gene involved in hearing loss is delivered to the inner ear hair cells of the subject.

The compositions and methods provided herein can also be used to treat a subject having or at risk of developing dizziness. In some embodiments, dizziness is associated with a vestibular disorder. Examples of vestibular disorders include, but are not limited to, benign paroxysmal positional vertigo (BPPV), labyrinthitis, vestibular neuritis, Meniere's disease, secondary endolymphatic hydrops, and perilymph fistula. Vestibular disorders also include superior canal dehiscence, acoustic neuroma, ototoxicity, enlarged vestibular aqueduct syndrome, and mal de débarquement.

Any of the methods of treating hearing loss or dizziness provided herein can be combined with other treatments for hearing loss or dizziness, for example, a hearing aid, administration of an effective amount of a corticosteroid, or exercises for treating vertigo, to name a few.

Throughout, treat, treating, and treatment refer to a method of reducing or delaying one or more effects or symptoms of hearing loss (e.g., trouble understanding speech, listening to television or radio at high volume, tinnitus, asking people to repeat themselves) or dizziness (e.g., loss of balance, fainting, double vision, confusion, slurred speech, numbness in arms or legs). The subject can be diagnosed with hearing loss or dizziness. Treatment can also refer to a method of reducing the underlying pathology rather than just the symptoms. The effect of the administration to the subject can have the effect of, but is not limited to, reducing one or more symptoms of the disease, a reduction in the severity of the disease, the complete ablation of the disease, or a delay in the onset or worsening of one or more symptoms. For example, a disclosed method is considered to be a treatment if there is at least about a 10% reduction in hearing loss or dizziness in a subject when compared to the subject prior to treatment or when compared to a control subject or control value. Thus, the reduction can be about a 10, 20, 30, 40, 50, 60, 70, 80, 90, 100%, or any amount of reduction in between. A reduction in hearing loss can also be a percentage improvement in hearing of at least about 5, 10, 20, 30, 40, 50, 60, 70, 80, 90, 100%, or any percentage in between these percentages. Methods for testing hearing in a subject are known in the art and include,

As used herein, by prevent, preventing, or prevention is meant a method of precluding, delaying, averting, obviating, forestalling, stopping, or hindering the onset, incidence, severity, or recurrence of a disease or disorder. For example, the disclosed method is considered to be a prevention if there is a reduction or delay in onset, incidence, severity, or recurrence of hearing loss or dizziness or one or more symptoms of hearing loss (e.g., trouble understanding speech, listening to television or radio at high volume, tinnitus, asking people to repeat themselves) or dizziness (e.g., loss of balance, fainting, double vision, confusion slurred speech, numbness in arms or legs) in a subject susceptible to hearing loss or dizziness compared to control subjects susceptible to hearing loss or dizziness that did not receive treatment. The reduction or delay in onset, incidence, severity, or recurrence of hearing loss or dizziness can be about a 10, 20, 30, 40, 50, 60, 70, 80, 90, 100%, or any amount of reduction in between.

As used throughout, by subject is meant an individual. The subject can be an adult subject or a pediatric subject. Pediatric subjects include subjects ranging in age from birth to eighteen years of age. Thus, pediatric subjects of less than about 10 years of age, five years of age, two years of age, one year of age, six months of age, three months of age, one month of age, one week of age or one day of age are also included as subjects. Preferably, the subject is a mammal such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical formulations are contemplated herein.

### Pharmaceutical Compositions

Provided herein is a pharmaceutical composition comprising any of the recombinant AAV virions described herein and a pharmaceutically acceptable carrier, diluent, excipient, or buffer. In some embodiments, the pharmaceutically acceptable carrier, diluent, excipient, or buffer is suitable for use in a subject, for example, a human. The pharmaceutical compositions can be delivered to a subject, so as to allow production of an expression product in an inner ear cell of the subject. Pharmaceutical compositions comprise sufficient genetic material that allows the recipient to produce an effective amount of an expression product that reduces or prevents inner hair cell damage. In some embodiments, the pharmaceutical compositions comprise sufficient genetic material that allows the recipient to produce an effective amount of an expression product that treats or prevents hearing loss and/or dizziness in a subject.

The compositions may be administered alone or in combination with at least one other agent, such as stabilizing compound, which may be administered in any sterile, biocompatible pharmaceutical carrier, including, but not limited to, saline, buffered saline, dextrose, and water. In some embodiments, the pharmaceutical compositions also contain a pharmaceutically acceptable excipient. Such excipients include any pharmaceutical agent that does not itself induce an immune response harmful to the individual receiving the composition, and which may be administered without undue toxicity. Pharmaceutically acceptable excipients include, but are not limited to, liquids such as water, saline, glycerol, sugars and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. The preparation of pharmaceutically acceptable carriers, excipients and formulations containing these materials is described in, e.g., Remington: The Science and Practice of Pharmacy, 22nd edition, Loyd V. Allen et al, editors, Pharmaceutical Press (2012).

Pharmaceutical formulations suitable for parenteral administration may be formulated in aqueous solutions, preferably in physiologically compatible buffers such as Hanks's solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

### Delivery Methods

The present disclosure provides a method of delivering an expression product to an inner ear hair cell in an individual, the method comprising administering to the individual a recombinant AAV virion as described above. The expression product can be a polypeptide, an antisense molecule, an interfering RNA or an aptamer, to name a few.

The term “effective amount,” as used throughout, is defined as any amount necessary to produce a desired physiologic response, for example, reducing or preventing inner ear hair cell damage. Effective amounts and schedules for administering the recombinant AAV virions described herein can be determined empirically and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, unwanted cell death, and the like. Generally, the dosage will vary with the type of inhibitor, the species, age, body weight, general health, sex and diet of the subject, the mode and time of administration and severity of the particular condition and can be determined by one of skill in the art. The dosage can be adjusted by the individual physician in the event of any contraindications. Dosages can vary and can be administered in one or more doses.

An effective amount of any of the recombinant AAV virions described herein will vary and can be determined by one of skill in the art through experimentation and/or clinical trials. For example, for in vivo injection, for example, injection directly into the inner ear of a subject, an effective dose can be from about 10⁶ to about 10¹⁵ recombinant rAAV virions, for example, from about 10⁸ to 10¹² recombinant AAV virions. For in vitro infection, an effective amount of recombinant virions to be delivered to cells can be from about 10⁶ to about 10¹⁵ of the recombinant AAV virions. Other effective dosages can be readily established by one of ordinary skill in the art through routine trials establishing dose response curves.

The compositions described herein are administered in a number of ways depending on whether local or systemic treatment is desired. The compositions are administered via any of several routes of administration, intravenously, intrathecally, intratypmanically, via round window administration, via semicircular canal delivery, or via stapedotomy. In some embodiments, the compositions are administered canalostomy into the posterior semicircular canal of the subject. Effective doses for any of the administration methods described herein can be extrapolated from dose-response curves derived from in vitro or animal model test systems.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### Examples

The following examples are provided by way of illustration only and not by way of limitation. Those of skill in the art will readily recognize a variety of non-critical parameters that could be changed or modified to yield essentially the same or similar results.

### Methods

### AAV Vector Construction

The AAV2.7m8-CAG-eGFP, AAV8BP2-CAG-eGFP, AAV2-CAG-eGFP, AAV2/8-CAG-eGFP, and Anc80L65-CAG-eGFP were produced by the Research Vector Core at the Center for Advanced Retinal and Ocular Therapeutics (University of Pennsylvania). The production method for these viruses are described in Ramachandran et al. (Evaluation of Dose and Safety of AAV7m8 and AAV8BP2 in the Non-Human Primate Retina. Hum Gene Ther 28, 154-167 (2017)). AAV-DJ-CAG-eGFP was purchased from Vector Biolabs (Malvern, Pa.). The concentration of viral stock solution was 1×10¹³ genome copies (G.C.) per ml for each virus.

### Animal Surgery

Animal surgery was approved by the Animal Care and Use Committee at the National Institute on Deafness and Other Communication Disorders (NIDCD ASP1378-18). Hypothermia was used to induce and maintain anesthesia in neonatal mice (P0-P5). Surgery was performed only on the left ear of each animal. The right ear served as a control. Inner ear gene delivery by posterior semicircular canal approach is described in (Isgrig, K., et al. Gene Therapy Restores Balance and Auditory Functions in a Mouse Model of Usher Syndrome. Mol Ther 25, 780-791 (2017)). Briefly, a post-auricular incision was made, and tissue was dissected to expose the posterior semicircular canal. Care was taken to avoid the facial nerve during the dissection. A Nanoliter Microinjection System (Nanoliter2000, World Precision Instruments, Sarasota, Fla.) was used in conjunction with a glass micropipette to load AAV-GFP into the glass micropipette. A total of 1 μl of AAV-eGFP was injected over approximately 40 seconds. Incision was closed with 5-0 vicryl sutures.

### Auditory Brainstem Response

Auditory brainstem response (ABR) testing was used to evaluate hearing sensitivity at ˜P30. Animals were anesthetized with ketamine (100 mg/kg) and xylazine (10 mg/kg) via intraperitoneal injections and placed on a warming pad inside a sound booth (ETS-Lindgren Acoustic Systems, Cedar Park, Tex.). The animal's temperature was maintained using a closed feedback loop and monitored using a rectal probe (CWE Incorporated, TC-1000, Ardmore, PN). Sub-dermal needle electrodes were inserted at the vertex (+) and test-ear mastoid (−) with a ground electrode under the contralateral ear. Stimulus generation and ABR recordings were completed using Tucker Davis Technologies hardware (RZ6 Multi I/O Processor, Tucker-Davis Technologies, Gainesville, Fla., USA) and software (BioSigRx, v.5.1). ABR thresholds were measured at 4, 8, 16, and 32 kHz using 3-ms, Blackman-gated tone pips presented at 29.9/sec with alternating stimulus polarity. At each stimulus level, 512-1024 responses were averaged. Thresholds were determined by visual inspection of the waveforms and were defined as the lowest stimulus level at which any wave could be reliably detected. A minimum of two waveforms was obtained at the threshold level to ensure repeatability of the response. Physiological results were analyzed for individual frequencies, and then averaged for each of these frequencies from 4 to 32 kHz.

### Circling Behavior

The circling behavior of mice that underwent inner ear gene delivery was quantified using optical tracking and the ANY-maze tracking software (version 4.96, Stoelting Co., Wood Dale, Ill.). A 38 cm×58 cm box was attached to a video camera (Fujinon YV5X2.7R4B-2 ⅓-inch 2.7-13.5 mm F1.3 Day/Night Aspherical Vari-Focal Lens). The ANY-maze video tracking software was set to track the head of mice placed within the box. Each mouse was placed into the box and allowed to acclimate to the new environment for 2 minutes. Complete rotations were recorded and quantified for the next 2 minutes, followed by a 1 minute “cool-down” period where rotations were not tracked. Each mouse was assessed three times, and the average was taken.

### Immunohistochemistry and Quantification

After completion of functional testing, mice were euthanized by CO₂ asphyxiation followed by decapitation. Temporal bones were harvested and fixed overnight with 4% paraformaldehyde followed by decalcification in 120 mM EDTA for 4 days. The vestibular organs and cochlear sensory epithelia were micro-dissected, blocked, and labeled with mouse anti-myosin 7a antibody to label hair cells (1:200, Proteus BioSciences, Ramona, Calif.), and mouse anti-acetylated tubulin antibody to label supporting cells (1:100, Sigma-Aldrich Corp., St. Louis, Mo.), chicken anti-GFP antibody (1:1000, Abcam, Cambridge, Mass.), and Hoechst stain (1:500, Life Technologies, Carlsbad, Calif.) to label nuclei. Primary and secondary antibodies were diluted in PBS. Images were obtained using a Zeiss LSM780 confocal microscope at 10× and 40× using z-stacks.

For hematoxylin and eosin (H&E) staining, tissues were first treated with a sucrose gradient (10-30% in PBS) and then were treated with a mixture of sucrose and embedding medium SCEM (Section-Lab Co Ltd, Japan). After freezing in liquid nitrogen, tissues were then sectioned at 10 μm thickness and H&E staining was done using the Hematoxylin & Eosin Stain Kit following the manufacturer's instructions (Vector Laboratories, Inc., Burlingame, Calif. USA).

For quantification of cochlear hair cell and supporting cell infection efficiency, two 40× images were taken at the apex, middle turn, and base of cochlea. The number of hair cells and supporting with GFP expression was counted and averaged at each location along the cochlea. Each 40× image contains ˜30 IHCs and ˜90 OHCs. The overall infection rate was calculated by averaging the infection rates obtained from the entire cochlea. For quantification of utricular hair cell infection efficiency, two 40× images (each containing ˜300 vestibular hair cells) were taken per utricle specimen and the number of hair cells with GFP expression was counted and averaged.

### Statistics

Student's t-test was used to assess differences in infection efficiency. Analysis of variance (ANOVA) was used to assess differences in ABR thresholds as well as circling behavior. Post-hoc analysis was performed using Scheffe's method. The p-value of <0.05 indicates statistical significance.

### Results

Many forms of hereditary hearing loss have mutations which affect the cochlear hair cells, the mechanosensory cells which allow for sound detection and processing. The infection patterns of three synthetic AAVs (AAV2.7m8, AAV8BP2, AAV-DJ) in the mouse inner ear were examined. AAV2.7m8 infects both IHCs and OHCs with high efficiency. In addition, AAV2.7m8 infects inner pillar cells and inner phalangeal cells with high efficiency. These results show that AAV2.7m8 is an excellent viral vector for inner ear gene therapy targeting cochlear hair cells and supporting cells. AAV2.7m8 greatly expand the applications for inner ear gene therapy.

AAV2.7m8 was generated using an in vivo-directed evolution approach where AAV libraries with diverse capsid protein modifications were screened for infection efficiency of mouse photoreceptor cells via intravitreal injection (Dalkara et al.) This vector contains a 10-amino acid peptide inserted at position 588 of the AAV2 capsid protein sequence, which is involved with AAV2 binding to its primary receptor, heparan sulfate proteoglycan (Dalkara et al.; and Khabou et al., Insight into the mechanisms of enhanced retinal transduction by the engineered AAV2 capsid variant −7m8. Biotechnol Bioeng 113, 2712-2724 (2016)). Similarly, AAV8BP2 was generated using an in vivo-directed evolution approach in which AAV libraries were screened for infection of mouse retinal bipolar cells via subretinal injection. This vector contains modifications at amino acids 585-594 of the AAV8 capsid protein sequence (Cronin, T., et al. Efficient transduction and optogenetic stimulation of retinal bipolar cells by a synthetic adeno-associated virus capsid and promoter. EMBO Mol Med 6, 1175-1190 (2014)). In addition to AAV2.7m8 and AAV8BP2, another synthetic AAV which has been used in various organ systems is AAV-DJ19. AAV-DJ was generated using DNA family shuffling technology where the viral capsid contains elements of various AAV serotypes (AAV2, 4, 5, 8, and 9) (Grimm, D., et al. In vitro and in vivo gene therapy vector evolution via multispecies interbreeding and retargeting of adeno-associated viruses. Journal of virology 82, 5887-5911 (2008)). AAV-DJ has been shown to infect hepatocytes, keratinocytes, neurons, and taste cells.

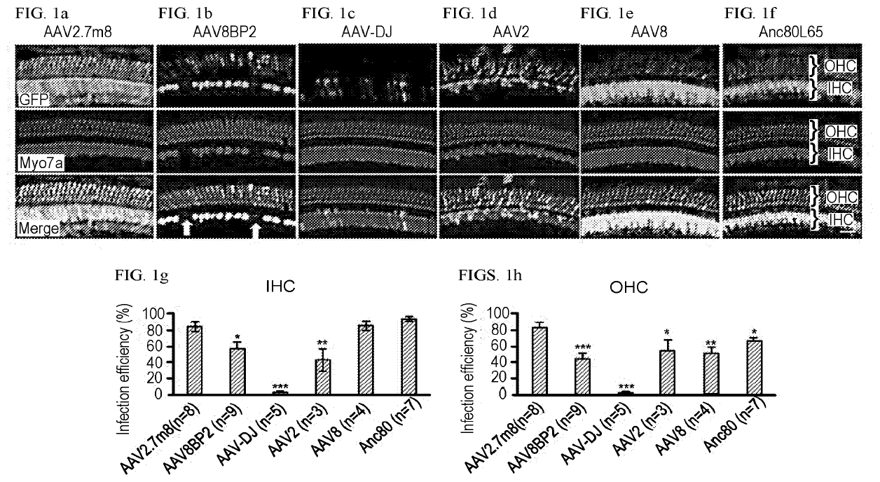

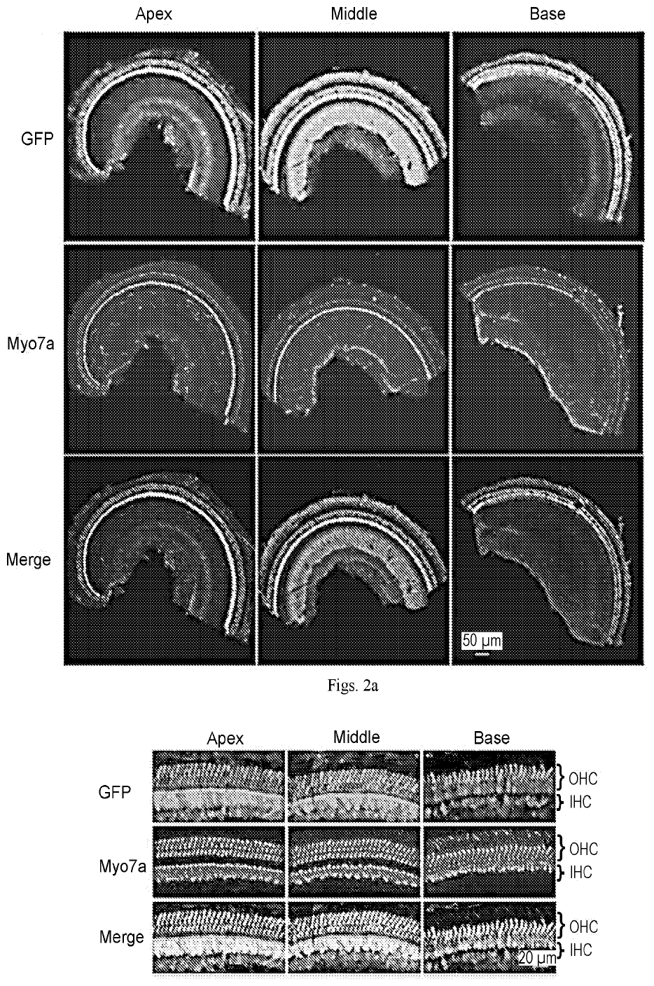

To assess the infection efficiency of synthetic AAVs in the mammalian inner ear, AAV2.7m8-GFP, AAV8BP2-GFP, and AAV-DJ-GFP were delivered to neonatal (P0-P5) mouse inner ears using the posterior semicircular canal approach. Posterior semicircular canal gene delivery allows viral vectors to effectively infect cells in the cochlea as well as vestibular organs (Isgrig, K., et al. Gene Therapy Restores Balance and Auditory Functions in a Mouse Model of Usher Syndrome. Mol Ther 25, 780-791 (2017); Tao, Y., et al. Delivery of Adeno-Associated Virus Vectors in Adult Mammalian Inner-Ear Cell Subtypes Without Auditory Dysfunction. Hum Gene Ther (2018); and Suzuki et al. Cochlear gene therapy with ancestral AAV in adult mice: complete transduction of inner hair cells without cochlear dysfunction. Scientific reports 7, 45524 (2017)). Infection efficiencies of AAV2-GFP and AAV8-GFP, two commonly used conventional AAVs from which AAV2.7m8 and AAV8BP2 are derived from respectively, as well as the synthetic AAV Anc80L65-GFP, were also examined using the same delivery approach as additional controls. Approximately 1×10¹⁰ genome copies (G.C.) were delivered into the inner ear of each animal. Hair cell infection efficiency was assessed by quantifying the percentage of hair cells (identified by anti-Myo7a antibody) with green fluorescent protein (GFP) expression. Examination of the cochlea 4 weeks after gene delivery revealed high levels of GFP in both IHCs and OHCs in mice that were injected with AAV2.7m8-GFP (n=8, FIG. 1, Table 1). The overall infection efficiency was 84.1±5.66% (mean±standard error) for IHC, and 83.1±6.17% for OHC. Mice injected with AAV8BP2-GFP (n=9, FIGS. 1a-1h, Table 1) had moderate to high levels of GFP expression in IHCs and OHCs. The overall infection efficiency was 55.7±9.53% for IHC, and 44.0±7.91% for OHC (p=0.016 and <0.001 for IHC and OHC respectively, when compared to AAV2.7m8). In contrast, mice injected with AAV-DJ-GFP (n=5, FIG. 1, Table 1) only had low levels of GFP expression in IHCs and OHCs. The overall infection efficiency was 1.63±1.27% for IHC, and 0.05±0.05% for OHC (p<0.001 for both IHC and OHC, when compared to AAV2.7m8).

TABLE 1

| | IHC | OHC | Utricle | IPC | IPhC |
|---|---|---|---|---|---|
| AAV2.7m8 | 84.1 | 83.1 | 27.5 | 86.1 | 61.4 |
| | (5.66) | (6.17) | (7.08) | (4.87) | (9.30) |
| AAV8BP2 | 55.7 | 44.0 | 34.2 | | |
| | (9.53) | (7.91) | (6.77) | 0 (0) | 0 (0) |
| AAV-DJ | 1.63 | 0.05 | 2.56 | 10.9 | |
| | (1.27) | (0.05) | (1.39) | (3.67) | 0 (0) |
| AAV2 | 43.6 | 54.5 | 32.4 | 60.3 | |
| | (13.5) | (12.7) | (6.52) | (7.96) | 0 (0) |
| AAV8 | 86.0 | 51.7 | 93.3 | 50.4 | |
| | (5.34) | (5.95) | (1.77) | (8.64) | 0 (0) |
| Anc80L65 | 94.0 | 67.0 | 67.7 | 75.3 | |
| | (3.20) | (3.81) | (2.68) | (4.94) | 0 (0) |

Infection efficiency of AAVs in various cell types in the inner ear. The infection rate (%) as well as the standard error (in parenthesis) are shown. IHC: inner hair cell. OHC: outer hair cell. IPC: inner pillar cell. IPhC: inner phalangeal cell.

Comparison of AAV2.7m8-GFP to conventional AAVs also showed superior cochlear hair cell infection efficiency, particularly with regard with OHCs. For AAV2-GFP (n=3, FIG. 1, Table 1), the overall infection efficiency was 43.6±13.5% for IHC, and 54.5±12.7% for OHC (p=0.003 and 0.03 for IHC and OHC respectively, when compared to AAV2.7m8). For AAV8-GFP (n=4, FIG. 1, Table 1), the overall infection efficiency was 86.0±5.34% for IHC, and 51.7±5.95% for OHC (p=0.84 and 0.003 for IHC and OHC respectively, when compared to AAV2.7m8).

Anc80L65 is a synthetic AAV which has been reported to infect both IHCs and OHCs. When Anc80L65-GFP was injected into neonatal mouse inner ears using posterior canal approach (n=7, FIG. 1, Table 1), the overall infection efficiency was 94.0±3.20% for IHC, and 67.0±3.81% for OHC. While the IHC infection efficiency is comparable between AAV2.7m8 and Anc80L65 (p=0.16), these data show that AAV2.7m8 is more capable at infecting OHCs compared to Anc80L65 (p=0.04).

Detailed examination of mice injected with AAV2.7m8-GFP (n=8) showed that AAV2.7m8 was able to infect hair cells throughout the entire cochlea (FIGS. 2a-2b). The IHC infection efficiency was 90.3±8.98% at the cochlear apex, 84.6±10.4% at the middle turn, and 77.5±10.8% at the cochlear base. The OHC infection efficiency was 89.0±9.53% at the cochlear apex, 85.2±10.9% at the middle turn, and 74.9±12.2% at the cochlear base. In four out of the eight mice that were injected with AAV2.7m8, the IHC and OHC infection rates were over 90% throughout the entire cochlea (FIG. 2). In one out of the eight mice that was injected with AAV2.7m8, the IHC and OHC infection rate was below 30%. This may have reflected inadvertent delivery of AAV2.7m8-GFP into the perilymph instead of endolymph. Taken together, these results indicate that AAV2.7m8 is a powerful viral vector which is capable of infecting both cochlear IHCs and OHCs with high efficiency.

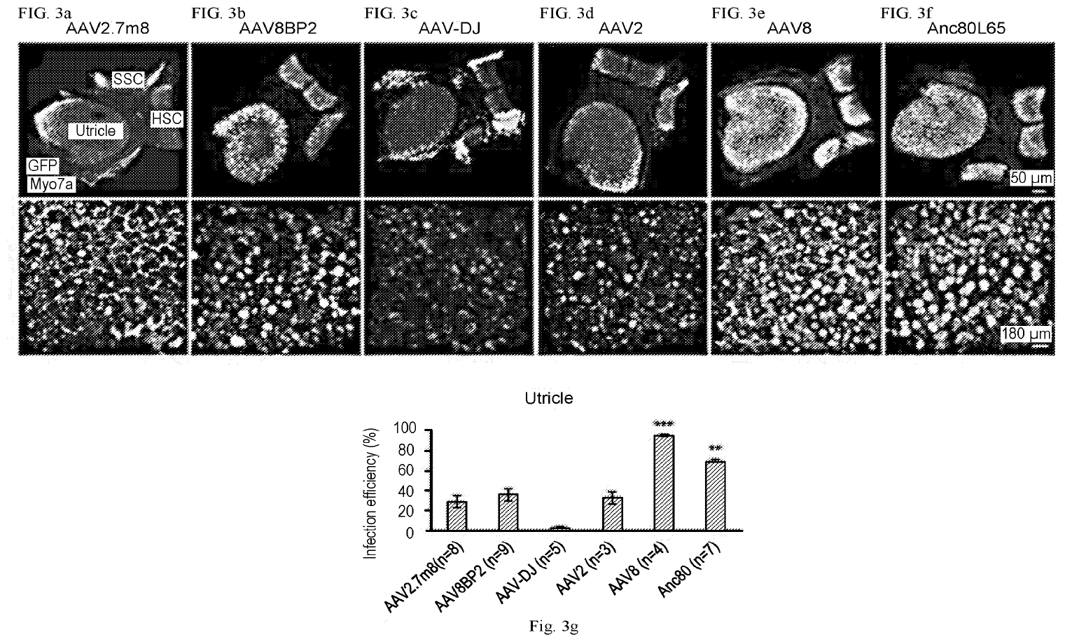

In addition to assessing hair cell infection efficiency of synthetic AAVs in the cochlea, the hair cell infection efficiency was also examined in the vestibular organs. When AAV2.7m8-GFP, AAV8BP2-GFP, and AAV-DJ-GFP were delivered to neonatal mouse inner ears, GFP was expressed in vestibular organs. Quantification of vestibular hair cell infection efficiency was done in the utricle (FIGS. 3a-3g, Table 1). The utricular hair cell infection efficiency was 27.5±7.08% for AAV2.7m8-GFP (n=8), 34.2±6.77% for AAV8BP2-GFP (n=9, p=0.63 compared to AAV2.7m8), and 2.56±1.39% for AAV-DJ-GFP (n=5, p=0.07 compared to AAV2.7m8). The vestibular hair cell infection efficiency of AAV2-GFP, AAV8-GFP, and Anc80L65-GFP were also examined in neonatal mouse utricles in vivo (FIG. 3, Table 1). The utricular hair cell infection efficiency was 32.4±6.52% for AAV2 (n=3, p=0.77 compared to AAV2.7m8), 93.3±1.77% for AAV8 (n=4, p<0.001 compared to AAV2.7m8), and 67.7±2.68% for Anc80L65 (n=7, p=0.002 compared to AAV2.7m8). These results indicate that AAV2.7m8 preferentially infects cochlear hair cells at much higher efficiency than vestibular hair cells.

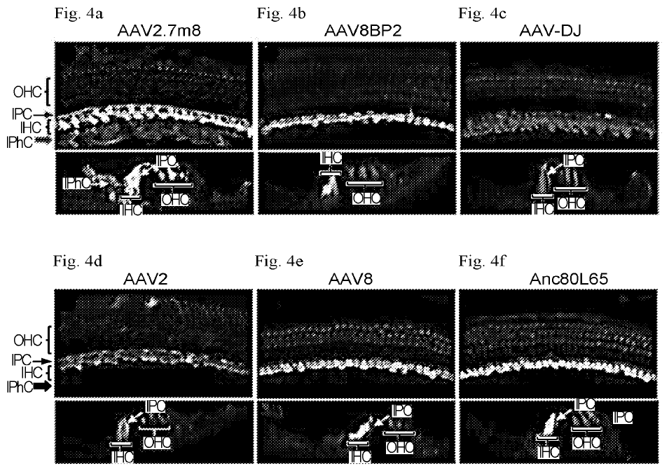

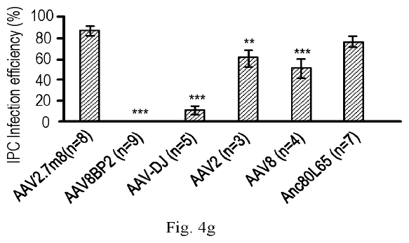

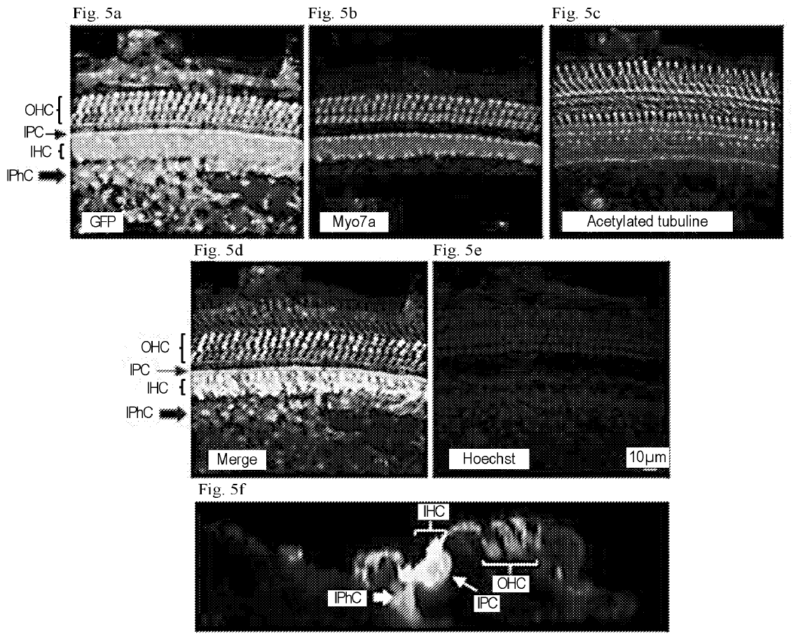

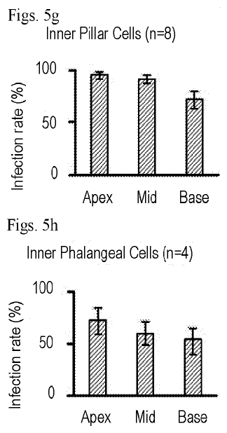

While cochlear hair cells have garnered the most attention as the targeted cell type in inner ear gene therapy studies, the glia-like supporting cells that surround hair cells are also important therapeutic targets for gene therapy. A specific subset of supporting cells, namely inner pillar cells, inner phalangeal cells, and the third row of Deiters cells, express Leucine-rich repeat-containing G-protein coupled receptor 5 (LGR5) and demonstrate progenitor cell-like properties that promote hair cell regeneration. When AAV2.7m8-GFP was delivered to neonatal mouse inner ears, GFP expression was seen in two of these LGR5+ supporting cell types—inner pillar cells and inner phalangeal cells (FIG. 4, FIGS. 5a-5g, Table 1). The overall inner pillar cell infection efficiency was 86.1±4.87% (94.7±3.11% at the apex, 91.3±3.80% at the middle turn, and 72.4±7.93% at the base, n=8). The overall inner phalangeal cell infection efficiency was 61.4±9.30% (72.0±12.5% at the apex, 60.0±11.1% at the middle turn, and 52.3±12.9% at the base, n=4). In contrast, mice injected with AAV8BP2 had no GFP expression in the inner pillar cells and inner phalangeal cells (FIGS. 4a-4g). Inner pillar cell infection was also seen mice injected with AAV-DJ-GFP (10.9±3.67%, n=5, p<0.001 compared to AAV2.7m8), AAV2-GFP (60.3±7.96%, n=3, p=0.007 compared to AAV2.7m8), AAV8-GFP (50.4±8.64%, n=4, p<0.001 compared to AAV2.7m8), and Anc80L65-GFP (75.3±4.94%, n=7, p=0.11). However, none of these AAVs infected inner phalangeal cells. These results suggest AAV2.7m8 is capable of infecting the subset of supporting cells (inner pillar cells and inner phalangeal cells) that are thought to be capable of promoting hair cell regeneration with high efficiency.

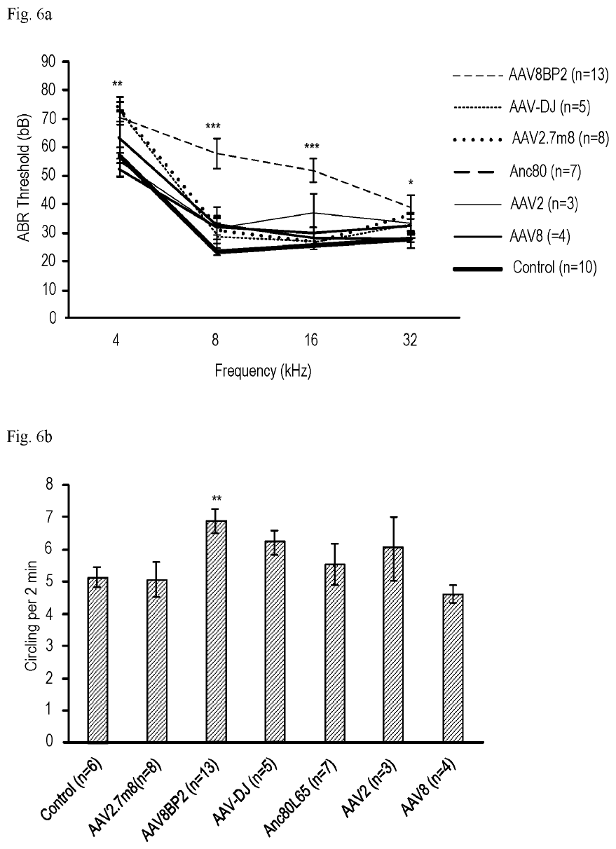

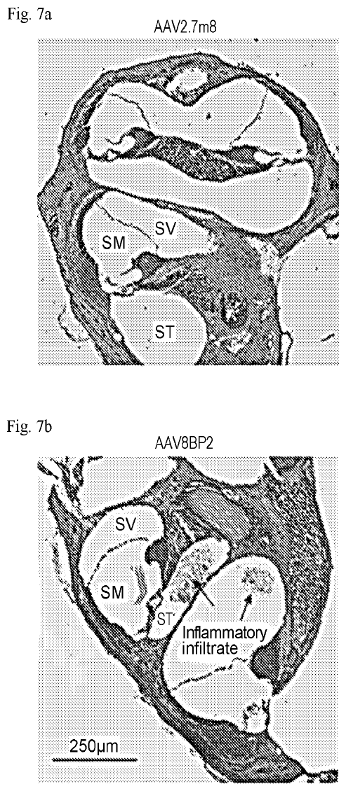

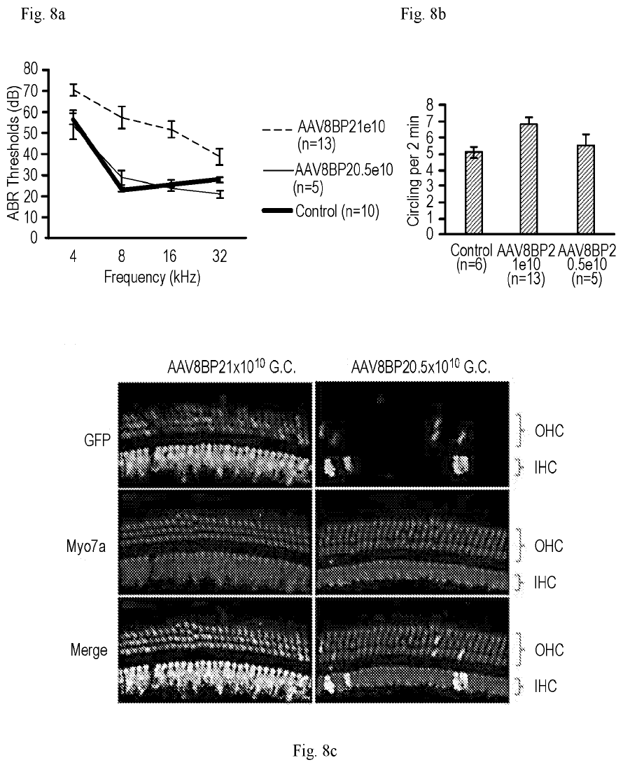

In order for inner ear gene therapy to be a viable treatment for hearing loss and vestibular dysfunction, the viral vector used should have minimal effect on normal auditory and vestibular functions. To assess whether inner ear delivery of synthetic AAVs had any effect on hearing, auditory brainstem responses (ABRs) were measured (FIGS. 6a-6b). Mice that underwent AAV2.7m8-GFP (n=8), AAV-DJ-GFP (n=5), AAV2-GFP (n=3), AAV8-GFP (n=4), and Anc80L65-GFP (n=7) injection showed no significant change in ABR thresholds compared to control mice that underwent no inner ear manipulation (p=0.09, 0.11, 0.25, 0.43, and 0.25, respectively, ANOVA). In contrast, mice that underwent AAV8BP2-GFP (n=13) injection showed a 10-25 dB ABR threshold elevation compared to control mice (p<0.001, ANOVA). Post-hoc comparisons using Scheffe's method showed statistically significant ABR threshold differences at 4 kHz, 8 kHz, 16 kHz, and 32 kHz (p=0.004, <0.001, <0.001, and 0.034 respectively). It is possible that AAV8BP2 is more immunogenic to the mouse inner ear, which leads to cochlear hair cell loss (FIG. 1) as well as ABR threshold elevation. Examination of the cochlea after AAV8BP2 injection revealed infiltration of inflammatory cells (FIGS. 7a-7b). When AAV8BP2-GFP was injected at half of the original concentration (0.5×1010 G.C.), the ABR thresholds were comparable to control mice (p=0.49, FIG. 8), but the IHC and OHC infection efficiency also decreased (43.2±8.36% and 23.3±5.41%, respectively, n=5), though the changes were not statistically significant (p=0.38 and 0.08 for IHC and OHC respectively).

Mice with vestibular dysfunction often exhibit circling behavior. To assess whether inner ear delivery of synthetic AAVs had any effect on the vestibular system, the circling behavior of injected mice was examined (FIG. 6). Control mice that did not undergo inner ear gene delivery circled 5.11±0.78 times per 2 minutes (n=6). The circling behavior of mice injected with AAV2.7m8-GFP (5.04±0.54 times per 2 minutes, n=8), AAV-DJ-GFP (6.20±0.36 times per 2 minutes, n=5), AAV2-GFP (6.00±1.02 times per 2 minutes, n=3), AAV8-GFP (4.58±0.28 times per 2 minutes, n=4), and Anc80L65-GFP (5.52±0.65 times per 2 minutes, n=7) was similar to non-injected control mice (p=0.92, 0.05, 0.31, 0.28, and 0.60 respectively, ANOVA). In contrast, mice that underwent AAV8BP2-GFP injection had a slight increase in circling (6.87±0.38 times per 2 minutes, p=0.009, n=13). Injection of AAV8BP2-GFP at half of the original concentration (0.5×1010 G.C.) resulted in no increase in circling behavior compared to control animals (5.47±0.77 times per 2 minutes, p=0.66, n=5, FIGS. 8a-8c). These results suggest that inner ear delivery of AAV2.7m8 is safe and resulted in little adverse effect in auditory and vestibular functions.

While several studies have shown that viral inner ear gene therapy improves auditory function in mouse models of hereditary hearing loss, the hearing recovery is often incomplete (Emptoz, A., et al. Local gene therapy durably restores vestibular function in a mouse model of Usher syndrome type 1G. Proc Natl Acad Sci USA 114, 9695-9700 (2017). One of the main drawbacks of conventional AAVs is that they infect the OHCs with low efficiency. Our results suggest that AAV2.7m8 is capable of infecting cochlear IHCs and OHCs with high efficiency. In fact, AAV2.7m8 infects OHCs at even higher efficiency than Anc80L65 when delivered through the posterior canal approach. It was found that AAV2.7m8 preferentially targeted the cochlear hair cells compared to vestibular hair cells. This is different from Anc80L65 which also infects vestibular hair cells with high efficiency (Landegger, L. D., et al. A synthetic AAV vector enables safe and efficient gene transfer to the mammalian inner ear. Nat Biotechnol 35, 280-284 (2017)). The predilection of AAV2.7m8 for targeting cochlear hair cells can be useful in studies where transgene expression is only desirable in the cochlea, and it can potentially minimize vestibular toxicity from unwanted transgene expression in the vestibular system.

Most inner ear gene therapy studies have focused on animal models of hereditary hearing loss. However, the prevalence of hereditary hearing loss is much lower than other types of hearing loss, such as age-related hearing loss (presbycusis) and noise-induced hearing loss. One strategy for applying gene therapy to treat presbycusis and noise-induced hearing loss is to induce hair cell regeneration. While the hair cells of non-mammalian animals (such as birds and zebrafish) are regenerated after damage, mammalian hair cells are not regenerated. The supporting cells are thought to serve as a source for hair cell regeneration. In the mammalian inner ear, the subset of supporting cells that are LGR5+ (inner pillar cells, inner phalangeal cells, and the third row of Deiters cells) have progenitor cell-like properties that promote hair cell regeneration. In order to utilize gene therapy to induce hair cell regeneration, one critical element is to have a viral vector which can effectively target this population of supporting cells. As shown herein, AAV2.7m8 effectively infects both the inner and outer hair cells in the cochlea. In addition, it also infects the types of supporting cells that have been shown by others to be LGR5+ (inner pillar cells and inner phalangeal cells) with very high efficiency. Taken together, these results demonstrate that AAV2.7m8 is a powerful viral vector that can greatly expand the applications for inner ear gene therapy.

1. A recombinant adeno-associated virus (AAV) virion comprising:
a) a modified AAV capsid protein, wherein the modified AAV capsid protein comprises a peptide insertion relative to a corresponding parental AAV capsid protein, wherein the peptide insertion comprises the amino acid sequence LGETTRP (SEQ ID NO: 1), wherein the insertion in the modified AAV capsid protein is between amino acids corresponding to amino acids 587 and 588 of AAV2-VP1; and
b) a heterologous nucleic acid that produces an expression product, wherein the expression product reduces hearing loss or dizziness.

2. The recombinant AAV virion of claim 1, wherein the expression product is a nucleic acid that decreases expression of a gene associated with hearing loss, wherein the gene is selected from the group consisting of DIAPH1, KCNQ4, GJB3, IFNLR1, GJB2, GJB6, MYH1, CEACAM16, GSDME/DFNA5. WFS1, LMX1A, TECTA, COCH, EYA4, MYO7A, COL11A2, POU4F3, MYH9, ACTG1, MYO6, SIX1, SLC17A8, REST, GRHL2, NLRP3, TMC1, COL11A1, CRYM, P2RX2, CCDC50, MIRN96, TJP2, TNC, SMAC/DIABLO, TBC1D24, CD164, OSBPL2, HOMER2, KITLG, MCM2, PTPRQ, DMXL2, MYO3A and PDE1C

3. The recombinant AAV virion of claim 1, wherein the expression product is a polypeptide that reduces hearing loss, wherein the polypeptide is selected from the group consisting of GJB2, GJB6, MYO7A, MYO15A, SLC26A4, TMIE, TMC1, TMPRSS3, OTOF, CDH23, GIPC3, STRC, USH1C, OTOG, TECTA, OTOA, PCDH15, RDX, GRXCR1, TRIOBP, CLDN14, MYO3A, WHRN, CDC14A, ESRRB, ESPN, MYO6, HGF, ILDR1, ADCY1, CIB2, MARVELD2, BDP1, COL11A2, PDZD7, PJVK, SLC22A4, SLC26A5, LRTOMT/COMT2, DCDC2, LHFPLS, S1PR2, PNPT1, BSND, MSRB3, SYNE4, LOXHD1, TPRN, GPSM2, PTPRQ, OTOGL, TBC1D24, ELMOD3, KARS, SERPINB6, CABP2, NARS2, MET, TSPEAR, TMEM132E, 123778442.1 PPIP5K2, GRXCR2, EPS8, CLIC5, FAM65B, DFNB32, EPS8L2, ROR1, WBP2, ESRP1, MPZL2, PRPS1, POU3F4, SMPX, AIFM1 and COL4A.

4. The recombinant AAV virion of claim 1, wherein the AAV virion is an AAV2 virion, an AAV5 virion, an AAV8 virion or an AAV9 virion.

5. The recombinant AAV virion of claim 1, wherein the AAV virion is an AAV2.7m8 virion.

6. The recombinant AAV virion of claim 2, wherein the nucleic acid that decreases expression of a gene associated with hearing loss is an interfering RNA.

7. The recombinant AAV virion of claim 6, wherein the interfering RNA is an antisense molecule, a short interfering RNA or an miRNA.

8. The recombinant AAV virion of claim 1, wherein the hearing loss is selected from the group consisting of age-related hearing loss, hereditary hearing loss, noise-induced hearing loss, disease-associated hearing loss and hearing loss resulting from trauma.

9. A method for treating or preventing inner ear hair cell damage in a subject comprising administering to the subject having inner ear hair cell damage or at risk of developing inner ear hair cell damage, an effective amount of the recombinant AAV virion of claim 1.

10. The method of claim 9, wherein the subject has or is at risk of developing age-related hearing loss, hereditary hearing loss, noise-induced hearing loss, disease-associated hearing loss and hearing loss resulting from trauma.

11. The method of claim 9, wherein the recombinant AAV virion infects inner hair cells and outer hair cells of the cochlea.

12. The method of claim 9, wherein the recombinant AAV virion infects glia-like supporting cells in the cochlea.

13. The method of claim 12, wherein the supporting cells are inner pillar cells or inner phalangeal cells.

14. The method of claim 9, wherein the recombinant AAV virion increases inner ear hair cell regeneration.

15. The method of claim 14, wherein the recombinant AAV virion increases cochlear hair cell regeneration

16. A method for treating or preventing hearing loss or dizziness in a subject, comprising administering to the subject having hearing loss or dizziness or at risk of developing hearing loss or dizziness, an effective amount of the recombinant AAV virion of claim 1.

17. The method of claim 16, wherein the subject has or is at risk of developing age-related hearing loss, hereditary hearing loss, noise-induced hearing loss, disease-associated hearing loss and hearing loss resulting from trauma.

18. The method of claim 16, wherein the recombinant AAV virion infects inner ear hair cells of the subject.

19. The method of claim 18, wherein the inner ear hair cells are inner and outer hair cells of the cochlea.

20. The method of claim 19, wherein the recombinant AAV virion infects glia-like supporting cells in the cochlea.

21. The method of claim 20, wherein the supporting cells are inner pillar cells or inner phalangeal cells.

22. The method of claim 16, wherein the recombinant AAV virion increases inner ear hair cell regeneration.

23. The method of claim 22, wherein the recombinant AAV virion increases cochlear hair cell regeneration

24. The method of claim 9, wherein the recombinant AAV virion is administered intravenously, intrathecally, intratypmanically, via round window administration, via semicircular canal delivery, or via stapedotomy.

25. The method of claim 24, wherein the recombinant AAV virion is administered via canalostomy into the posterior semicircular canal of the subject.


SEQUENCE LISTING (2 sequences)

SEQ ID NO: 1 - protein, 7 aa, Artificial sequence
  Synthetic construct
LGETTRP

SEQ ID NO: 2 - protein, 735 aa, Artificial sequence [sequence omitted]